(19) United States
(12) Patent Application Publication
(10) Pub. No.: US 2021/0228307 A1
(43) Pub. Date: Jul. 29, 2021

(54) SURGICAL OBSERVATION SYSTEM

(71) Applicants: Keio University, Tokyo (JP); Mitaka Kohki Co., Ltd., Tokyo (JP)

(72) Inventors: Eiji KOBAYASHI, Wakayama (JP); Shinji UEMOTO, Kyoto (JP); Shintaro YAGI, Kyoto (JP); Katsuyuki NAKAMURA, Tokyo (JP)

(73) Assignees: Keio University, Tokyo (JP); Mitaka Kohki Co., Ltd., Tokyo (JP)

(21) Appl. No.: 17/056,205

(22) PCT Filed: May 23, 2019
(86) PCT No.: PCT/JP2019/020425

§ 371 Date: Nov. 17, 2020

(30) Foreign Application Priority Data
May 24, 2018 (JP) 2018-099341

(51) Int. Cl. A61B 90/00

(52) U.S. Cl. CPC A61B 90/361; A61B 2560/02; A61B 90/37

(57) ABSTRACT

A monitor is arranged on the opposite side of a surgery table to a main doctor so that an assistant is able to view a surgery area directly without being blocked by the monitor. A camera has a working distance of more than or equal to 60 cm and is therefore above a view field for the main doctor to view the monitor, so that the visibility of the monitor is not hindered. The working distance is not more than 100 cm so that the resolution of the camera is not lowered too much.

### TECHNICAL FIELD

The present invention relates to a surgical observation system.

### BACKGROUND ART

There is known a surgical observation system that, in a state a doctor being positioned on one side of a surgery table, supports a monitor above the surgery table and also a camera slightly before the monitor right above a surgical site and displays, on the monitor, a stereoscopic image of the surgical site picked up by the camera.

The doctor is able to observe, in a state with his or her head raised (in a head-up state), the stereoscopic image of the surgical site displayed on the monitor and carry out an operation. As a result, unlike an operation conducted with a microscope, the doctor is not forced to take a posture of placing his or her eyes on eyepieces of the microscope but is able to take a comfortable position to carry out the operation, so that fatigue of the doctor is remarkably reduced. A related art is disclosed in, for example, Japanese Unexamined Patent Application Publication No. H11-318936 (Patent Literature 1).

### SUMMARY OF INVENTION

### Problems to be Solved by Invention

However, according to such a related art, the monitor is supported above the surgery table, and therefore, an assistant and the like positioned on the other side of the surgery table are blocked by the monitor and are unable to see a condition of the surgical site so that they are unable to carry out sufficient supportive work on the surgical site.

In addition, in recent years, to observe a surgical site not only within a specific magnification range but also from a low magnification for widely observing the surgical site and its periphery to an intermediate magnification using a loupe and further to a high magnification using a microscope so as to cope with every surgical situation, there is a requirement for observing, with a single monitor, a wide magnification range continuously and seamlessly (borderlessly).

### Means to Solve Problems

The present invention has been made in consideration of the above-mentioned circumstances and provides a surgical observation system capable of allowing an assistant and the like positioned on the opposite side of a surgery table to surely support an operation and realizing, with a single monitor, a continuous and seamless (borderless) observation in a wide magnification range.

According to a first technical aspect of the present invention, the surgical observation system includes a camera that is arranged above a surgery table in a state securing a predetermined working distance to a surgical site, is provided with an optical system and image sensor for picking up a stereoscopic image of the surgical site, and outputs an image signal related to the picked-up stereoscopic image and a monitor that is arranged, with a main doctor being positioned on one side of the surgery table, on the other side of the surgery table at a position higher than the surgery table, to receive the image signal outputted from the camera and display a stereoscopic image based on the image signal. It is characterized in that the working distance (WD) of the camera satisfies a below-mentioned conditional equation:

60 cm<=WD<=100 cm (1).

According to a second technical aspect of the present invention, it is characterized in that the optical system includes an objective optical system and a variable-power optical system and that a total magnification (TM) obtained from an optical magnification (OM) of the optical system and a monitor magnification (IM) of the stereoscopic image displayed on the monitor satisfies a below-mentioned condition depending on a choice of the working distance (WD) and a choice of the size of the monitor:

3 times<=TM<=44 times (2)

where TM=OM×IM and IM=diagonal length of the monitor/diagonal length of the image sensor.

According to a third technical aspect of the present invention, it is characterized in that the variable-power optical system has a variable power capacity to vary between a minimum total magnification and a maximum total magnification at a chosen working distance and monitor size in a time (MS) that satisfies a below-mentioned condition:

one second<=MS<=two seconds (3).

According to a fourth technical aspect of the present invention, it is characterized in that the camera includes two kinds of image sensors, one for visible light and the other for invisible light and that visible-light and invisible-light stereoscopic images can selectively and simultaneously overlappingly be displayed on the monitor.

### BRIEF DESCRIPTION OF DRAWINGS

FIG. 1 is a perspective view illustrating a surgical observation system.

FIG. 2 is an explanatory view illustrating positional relationships of a camera and monitor with respect to a surgery table.

FIG. 3 is a perspective view illustrating an internal structure of the camera.

FIG. 4 is a front view illustrating the internal structure of the camera.

FIG. 5 is a side view illustrating the internal structure of the camera.

FIG. 6 is a graph illustrating a speed of changing a total magnification.

### MODE OF IMPLEMENTING INVENTION

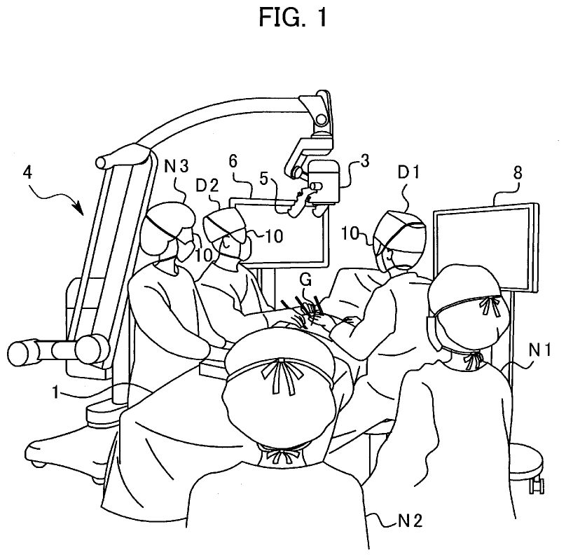

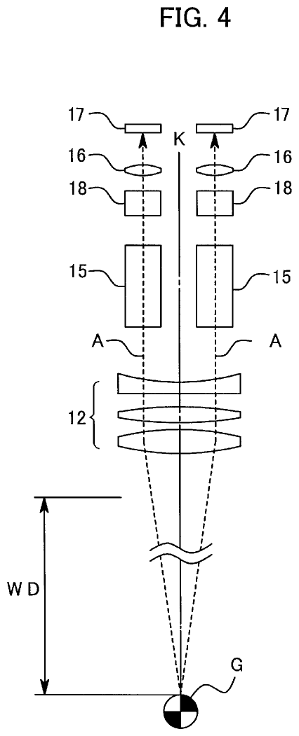

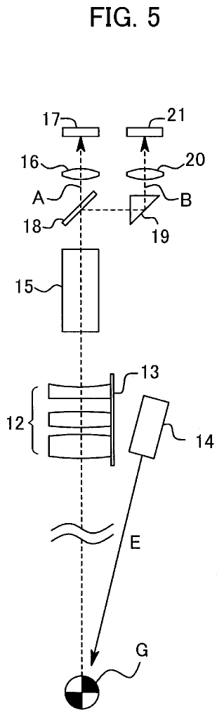

FIGS. 1 to 6 are views illustrating a preferred embodiment of the present invention.

On a surgery table 1, a patient 2 is laid, and at the abdomen thereof, there is a surgical site G to be operated. On this side of the surgery table 1, there are a main doctor D1 to conduct the operation and his or her two assistants N1 and N2. On the other side of the surgery table 1, there are a sub-doctor D2 to assist the operation and his or her assistant N3.

Above the surgical site G, a camera 3 is supported with a stand device 4. The position of the camera 3 can vertically be moved by moving the stand device 4. A working distance WD of the camera 3 (a distance from the surgical site G to a lower end of the camera 3) is 60 to 100 cm, and in this embodiment, the camera 3 is supported at a position of 100 cm. The camera 3 is of 4K and is able to pick up a stereoscopic image of visible light and a stereoscopic image of invisible light such as infrared fluorescence (hereunder, fluorescence) and the like.

The camera 3 is provided with a left-and-right pair of control handles 5. The control handles 5 are grasped to vertically move the camera 3 or change the direction thereof. Also, the control handles 5 are provided with a plurality of buttons. By operating the buttons, it is possible to achieve a focal point adjustment, magnification change, visible light image and fluorescence image switching, visible light image and fluorescence image overlapping, and the like.

Adjacent to the sub-doctor D2 on the opposite side of the surgery table 1, there is arranged a liquid-crystal-panel-type monitor 6 of 55-inch size capable of displaying a stereoscopic image in 4K. This monitor 6 is for the main doctor D1 and his or her assistants N1 and N2 and the main doctor D1 is able to confirm, from this side across the surgery table 1, a stereoscopic image displayed on the monitor 6 and conduct the operation.

The monitor 6, in order to be easily seen from the main doctor D1, is arranged at a position h1 higher by 20 cm than the surgery table 1. Since a height h2 of the 55-inch monitor 6 is about 70 cm, an upper end part of the monitor 6 is at a height position of about 90 cm from the surgery table 1.

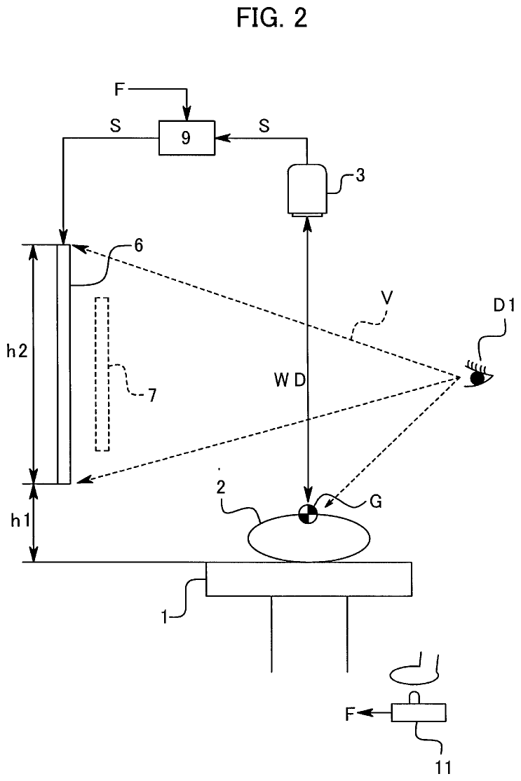

The size of the monitor 6 used for surgery is generally, as a type set on the opposite side of the surgery table 1 like this embodiment, 32 to 55 inches. The 55-inch size among the generally used sizes corresponds to a large size. A 32-inch monitor 7 corresponds to a small size and is illustrated for reference with a dotted line in FIG. 2.

On a head side of the patient 2 on the surgery table 1, another monitor 8 is arranged. This monitor 8 is for the sub-doctor D2 and his or her assistant N3, is in the same size as the monitor 6 for the main doctor D1, and is arranged at the same height.

The camera 3 and monitors 6 and 8 are connected to each other through a controller 9. The camera 3 outputs, as an image signal S, a picked-up stereoscopic image of the surgical site G to the controller 9. The image signal S is outputted from the controller 9 to the monitors 6 and 8, and the monitors 6 and 8 display the stereoscopic image based on the image signal S. The main doctor D1 and assistants N1 and N2 each wear special glasses 10 so that they are able to observe the stereoscopic image displayed on the monitor 6. The sub-doctor D2 and assistant N3 also wear the special glasses 10 so that they are able to observe the stereoscopic image displayed on the other monitor 8.

Since the sub-doctor D2 and assistant N3 are positioned on the same side as the monitor 6 with respect to the surgery table 1, they are able to directly see the surgical site G without being blocked by the monitor 6, thereby surely carrying out supportive work of the operation.

The controller 9 is also connected to a foot switch 11 under the foot of the main doctor D1. This foot switch 11 allows control to be performed like the buttons of the control handles 5 and outputs a signal F to the controller 9.

The upper end position of the monitor 6 arranged on the opposite side of the surgery table 1 is at a height of about 90 cm. Since the camera 3 has the working distance WD of 100 cm and is supported at a position of 100 cm above the surgical site G, the camera 3 never enters in a view field V in which the eyes of the main doctor D1 see the monitor 6. Since the camera 3 is upwardly shifted off the view field V for seeing the monitor 6, a visibility of the monitor 6 is never hindered.

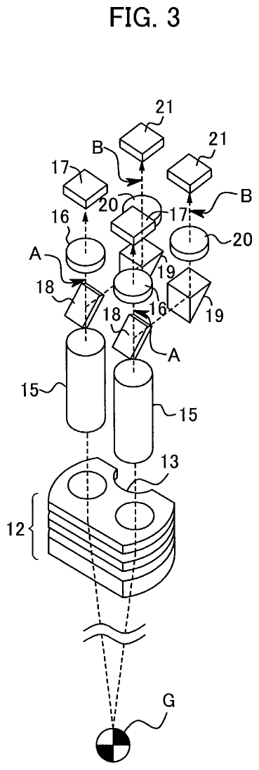

A structure of the camera 3 will be explained with reference to FIGS. 3 to 5.

To achieve stereoscopic photographing, the camera 3 internally forms left and right two optical paths. At a lower part of the camera 3, there is arranged an objective optical system 12 formed with three lenses. On an optical axis K of the objective optical system 12, the surgical site G to be observed is positioned. The objective optical system 12 is able to change, by moving some of the lenses, a focal length from 60 cm to 100 cm. If the working distance WD of the camera 3 is below 60 cm and if the monitor 6 is of large size like the embodiment, there is a risk that the camera 3 comes into the view field V of the main doctor D1. Further, the larger the working distance WD, the lower the resolution of the camera 3 decreases, and therefore, it must not exceed 100 cm at the maximum.

The objective optical system 12 has a shape with front and back thereof being cut, and on the back side thereof, there is formed a semicircular cut part 13 partitioned with a douser. The cut part 13 is provided with a lighting unit 14. The lighting unit 14 is arranged at a predetermined angle relative to the optical axis K of the objective optical system 12.

The lighting unit 14 incorporates an LED light source and is able to selectively and simultaneously emit, as illuminating light E, both visible light and excitation light of a specific wavelength for fluorescence. By a not-illustrated rotation mechanism, the angle of the lighting unit 14 is changed according to a focal distance of the objective optical system 12, so that the illuminating light E is always oriented to the center of the surgical site G. Although this embodiment arranges the lighting unit 14 inside the camera 3, it is possible to arrange the same outside to guide the illuminating light E through an optical fiber into the camera 3, and then, emit the light toward the surgical site G.

On the objective optical system 12 within the range thereof, there are arranged two variable-power optical systems 15 in a left-and-right pair. Due to the variable-power optical systems 15 and objective optical system 12, the camera 3 provides, when the working distance WD is 60 cm, an optical magnification (OM) of 0.03 to 0.19 times, and when the working distance WD is 100 cm, an optical magnification (OM) of 0.02 to 0.12 times. By changing the working distance WD between 60 and 100 cm, it provides in total an optical magnification (OM) of 0.02 to 0.19 times. Since the working distance WD is large and the focal length of the objective optical system 12 is long, the optical magnification becomes small.

Passing through the objective optical system 12 and through the two variable-power optical systems 15, there are main optical paths A. The main optical paths A pass through imaging lenses 16 and are guided to image sensors 17 for visible light. The image sensors 17 each are a ⅓-inch size 4K CCD image sensor.

Between the variable-power optical systems 15 and the imaging lenses 16, there are arranged dichroic mirrors serving as optical branching units 18. With the optical branching units 18, the visible light is passed as the main optical paths A and the fluorescence (infrared light) is branched (reflected) to form optical sub-paths B. The optical sub-paths B are reflected by prisms 19, passed through imaging lenses 20, and guided to fluorescence image sensors 21. The image sensors 21 each are also a ⅓-inch size 4K CCD image sensor. In this way, other than the left-and-right pair of main optical paths A, the left-and-right pair of optical sub-paths B are formed so that a fluorescence stereoscopic image is able to be picked up by the image sensors 21 and displayed on the monitors 6 and 8.

Since the size of the monitor 6 is 55 inches (a diagonal length of 1397 mm) and the size of each of the image sensors 17 and 21 is ⅓ inches (a diagonal length of 6 mm), a monitor magnification (IM) becomes 232.8 times. In the case of the small 32-inch monitor 6, a diagonal length is 813 mm, and therefore, the monitor magnification (IM) is 135.5 times.

A total magnification TM (TM=OM×IM) of the surgical observation system will be 3 to 44 times if the working distance WD is in the range of 60 to 100 cm and if the size of the monitor 6 is in the general range of small (32 inches) to large (55 inches). By choosing the working distance WD and monitor size, a magnification of 3 to 44 times is obtainable, and therefore, any surgery table condition can be coped with. In addition, the surgical site can borderlessly be observed from the minimum total magnification up to the maximum total magnification determined by the working distance WD and the size of the monitors 6 and 8.

According to this embodiment, the working distance WD is 100 cm and the monitor 6 is chosen to be 55 inches, and therefore, the total magnification is 4.7 to 27.9. Then, between the minimum total magnification (4.7 times) and the maximum total magnification (27.9 times), the variable-power optical systems 15 of the camera 3 can be set to change in one to two seconds.

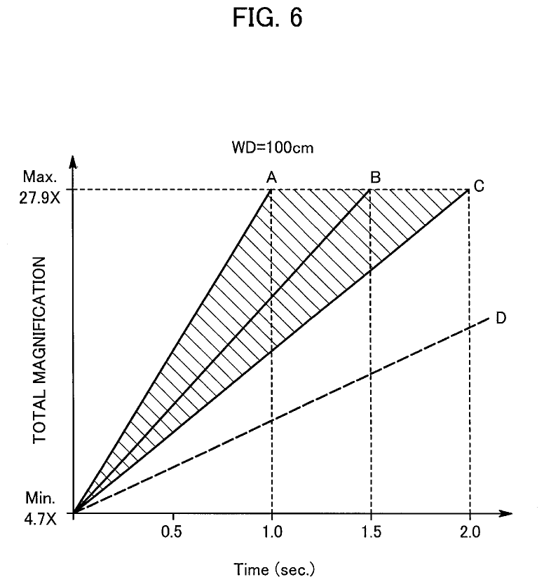

FIG. 6 is a graph illustrating the variable power capacity. The camera 3 is able to choose, with the control handles 5, a mode A capable of changing from the minimum total magnification to the maximum total magnification in one second, a mode B capable of conducting the same change in 1.5 seconds, and a mode C capable of conducting the same change in 2.0 seconds. A mode D in FIG. 6 is a conventional speed, i.e., a variable power speed of conducting the same change in 5.0 seconds. In the mode D, changing from the minimum total magnification to the maximum total magnification takes a time to deteriorate operability. According to the embodiment, between one and two seconds, the change is completed in a short time in an optimum mode chosen by the main doctor D1, and therefore, in various surgical phases, a stereoscopic image of a required total magnification can instantaneously be displayed on the monitors 6 and 8 to realize an excellent operability.

The embodiment employs the optical branching units 18 to separate visible light and infrared light (invisible light). Not limited by this, it is possible to branch light into two or more spectral ranges and selectively and overlappingly display them on the monitors 6 and 8.

According to the first technical aspect of the present invention, a monitor is arranged opposite to a main doctor with a surgery table interposed between them, and therefore, an assistant and the like positioned on the same side as the monitor with respect to the surgery table are able to directly see a surgical site without bothered by the monitor and surely carry out auxiliary work of an operation.

Also, to observe the surgical site borderlessly from a low magnification to a high magnification, a large monitor is required in some cases. Even if the monitor is of large size to expand a vertical view field in which the main doctor sees the monitor, the working distance of a camera is 60 cm or over so that the position of the camera is upwardly off the monitor seeing view field, and therefore, visibility of the monitor is never hindered. Also, since the working distance is 100 cm or below, the resolution of the camera is never decreased too low.

According to the second technical aspect of the present invention, in the case of a working distance of 60 to 100 cm and by choosing a monitor size, a total magnification (optical magnification multiplied by monitor magnification) will be 3 to 44 times, to enable to cope with any surgical situation. In addition, a surgical site will borderlessly be observable from a minimum total magnification to a maximum total magnification determined by the working distance and monitor size. Since the monitor is on the opposite side of the surgery table, the main doctor is also able to observe the surgical site with the naked eye.

According to the third technical aspect of the present invention, it is possible to change in one to two seconds between a minimum total magnification and a maximum total magnification determined by a chosen working distance and monitor size. Accordingly, even if the magnification range from the minimum total magnification to the maximum total magnification is fully used during surgery, the magnification can be changed in a short time to realize an excellent operability.

According to the fourth technical aspect of the present invention, a visible-light stereoscopic image can be switched to an invisible-light stereoscopic image, or the invisible-light stereoscopic image can be overlaid in the visible-light stereoscopic image, to enable an operation using invisible fluorescence to be coped with.

### (United States Designation)

In connection with United States designation, this international patent application claims the benefit of priority under 35 U.S.C. 119(a) to Japanese Patent Application No. 2018-099341 filed on May 24, 2018 whose disclosed contents are incorporated herein by reference.

1. A surgical observation system comprising:
a camera that is arranged above a surgery table in a state securing a predetermined working distance to a surgical site, being provided with an optical system and image sensor for picking up a stereoscopic image of the surgical site, and outputting an image signal related to the picked-up stereoscopic image, wherein a main doctor being positioned on one side of the surgery table; and
a monitor receiving the image signal outputted from the camera and display a stereoscopic image based on the image signal and being arranged on the other side of the surgery table at a position higher than the surgery table, wherein
the working distance (WD) of the camera satisfies a below-mentioned conditional relationship:
60 cm<=WD<=100 cm (1).

2. The surgical observation system as set forth in claim 1, wherein:
the optical system includes an objective optical system and a variable-power optical system; and
a total magnification (TM) obtained from an optical magnification (OM) of the optical system and a monitor magnification (IM) of the stereoscopic image displayed on the monitor satisfies a below-mentioned relationship depending on a choice of the working distance (WD) and a choice of the size of the monitor:
3 times<=TM<=44 times (2)
where TM=OM×IM and IM is a diagonal length of the monitor divided by a diagonal length of the image sensor.

3. The surgical observation system as set forth in claim 2, wherein the variable-power optical system has a variable power capacity to vary between a minimum total magnification and a maximum total magnification at a chosen working distance and monitor size in a time (MS) that satisfies a below-mentioned relationship:
one second<=MS<=two seconds (3).

4. The surgical observation system as set forth in claim 1, wherein:
the camera includes two kinds of image sensors, one for visible light and the other for invisible light; and
visible-light and invisible-light stereoscopic images can selectively and simultaneously overlappingly be displayed on the monitor.
